(19) United States
(12) Patent Application Publication
(10) Pub. No.: US 2026/0131099 A1
(43) Pub. Date: May 14, 2026

(54) Integrated CPAP Mask with ETCO2 Filtration for Enhanced Respiratory Monitoring and Treatment in Emergency Medical Settings

(71) Applicant: Austin Bates, Manhattan Beach, CA (US)

(72) Inventor: Austin Bates, Manhattan Beach, CA (US)

(21) Appl. No.: 18/940,838

(22) Filed: Nov. 8, 2024

(51) Int. Cl. A61M 16/06; A61M 16/00; A61M 16/08

(52) U.S. Cl. CPC A61M 16/0683; A61M 16/0003; A61M 16/0051; A61M 16/024; A61M 16/0875; A61M 2202/0007; A61M 2202/0208; A61M 2205/02; A61M 2205/18; A61M 2205/3341; A61M 2205/3576; A61M 2205/50; A61M 2209/088; A61M 2230/40

(57) ABSTRACT

The present invention relates to a Continuous Positive Airway Pressure (CPAP) mask with integrated End-Tidal Carbon Dioxide (ETCO2) monitoring for use in emergency medical services (EMS) and other healthcare settings. This device enables healthcare providers to simultaneously administer CPAP therapy while continuously monitoring ETCO2 levels in patients experiencing respiratory distress. The integrated design eliminates the need for multiple masks and oxygen sources, streamlining the delivery of respiratory care and improving patient comfort. The CPAP mask that captures exhaled carbon dioxide, providing real-time data transmission to an external monitoring system. By combining both respiratory therapy and carbon dioxide monitoring in a single device, this invention simplifies application, enhances treatment efficiency, and reduces equipment complexity, making it ideal for use in pre-hospital and non-clinical environments. The system further optimizes patient care through improved mask seal integrity, easier provider training, and cost savings for healthcare providers.

### BACKGROUND OF THE INVENTION

### Field of Invention

The present invention relates to the field of respiratory therapy and monitoring, specifically to a Continuous Positive Airway Pressure (CPAP) mask with integrated End-Tidal Carbon Dioxide (ETCO2) filtration. This invention is designed for use in emergency medical services (EMS) and other healthcare settings to provide simultaneous respiratory treatment and ETCO2 monitoring in a single, streamlined device. The invention addresses the limitations of current CPAP systems by combining respiratory support and carbon dioxide monitoring into one unit, enhancing ease of use, patient comfort, and treatment efficiency.

### BRIEF SUMMARY OF THE INVENTION

The present invention provides a Continuous Positive Airway Pressure (CPAP) mask with integrated End-Tidal Carbon Dioxide (ETCO2) filtration, enabling medical providers to administer respiratory support while simultaneously monitoring ETCO2 levels in patients. This invention eliminates the need for two separate masks and multiple oxygen sources by combining both CPAP therapy and ETCO2 monitoring into a single, efficient device. The integrated design reduces bulk, improves the mask seal, and simplifies application, resulting in enhanced patient comfort, more effective treatment, and ease of use for healthcare providers, particularly in emergency medical services (EMS) and non-clinical environments. By removing the need for separate equipment, this invention optimizes patient care, reduces potential fail points, and lowers equipment costs for healthcare providers.

### BRIEF DESCRIPTION OF THE FIGURES

FIG. 1: illustrates a process according to the present invention.

FIG. 2: illustrates the CPAP mask of the present invention.

FIG. 3: illustrates a user wearing the CPAP mask of the present invention.

### DETAILED DESCRIPTION

The present invention pertains to a novel system for providing continuous positive airway pressure (CPAP) therapy in conjunction with integrated End-Tidal Carbon Dioxide (ETCO2) monitoring, enabling healthcare providers to offer both respiratory support and real-time ventilation monitoring using a single device. This is particularly advantageous in emergency medical services (EMS) settings and non-clinical environments where quick and effective treatment is essential. The system alleviates the need for separate CPAP masks and ETCO2 monitoring systems, significantly enhancing patient care while simplifying the workflow for medical providers.

### System Architecture

### CPAP Mask Design

The primary component of the system is a Continuous Positive Airway Pressure (CPAP) mask that is engineered to deliver a constant flow of pressurized air to patients experiencing respiratory distress, such as those with chronic obstructive pulmonary disease (COPD), asthma, or congestive heart failure. The CPAP mask maintains positive airway pressure to prevent airway collapse during both inhalation and exhalation, improving oxygenation and reducing the work of breathing.

### Integrated ETCO2 Filtration Mechanism

Incorporated within the CPAP mask is a sophisticated ETCO2 filtration and monitoring mechanism, which continuously measures the concentration of carbon dioxide (CO2) exhaled by the patient. The ETCO2 levels are a direct reflection of the patient's ventilatory status and provide critical information for diagnosing and managing respiratory conditions. ETCO2 monitoring is essential for identifying conditions such as hypoventilation, hyperventilation, and perfusion abnormalities, making it an indispensable tool in emergency care settings.

The filtration mechanism uses advanced sensor technology to capture exhaled gasses directly from the patient's airway. The sensors are embedded within the CPAP mask in a way that ensures accurate, real-time measurement of ETCO2 without obstructing airflow or interfering with the pressure mechanics of the CPAP system. The sensor technology is highly sensitive, capable of detecting subtle changes in CO2 concentration with high accuracy. It is positioned to monitor both the inspiratory and expiratory phases of breathing, allowing for a full cycle of respiratory analysis.

### Data Transmission and Display

### Oxygen Supply and Tubing Configuration

The invention reduces the complexity of traditional systems by requiring only a single oxygen source for both CPAP therapy and ETCO2 monitoring. In existing configurations, separate oxygen lines are necessary for each system, often leading to a cumbersome and tangled arrangement of tubing. The integrated design eliminates the need for multiple oxygen sources, simplifying setup and reducing the risk of errors during treatment.

### Patient Comfort and Ergonomic Considerations

Recognizing that many patients requiring CPAP therapy are already in significant distress, this invention places considerable emphasis on comfort. The CPAP mask's ergonomic design ensures that it can be applied quickly and adjusted easily to fit a wide range of facial structures. The mask's interface is padded with silicone-based materials, which mold to the patient's face and reduce pressure points, thereby increasing tolerance for long-term use.

The system is designed to be intuitive and easy for EMS personnel to apply, ensuring rapid deployment even under duress, ensuring that no additional external devices or nasal cannulas are necessary. This reduces the patient's discomfort and anxiety, which are often exacerbated by bulky or unfamiliar equipment in emergency situations.

### Operational Process

### Step 1: Application of the CPAP Mask

Upon assessing the patient's need for respiratory support, the healthcare provider selects the integrated CPAP mask. The machine is turned on and the mask is applied directly over the patient's nose and mouth, ensuring that the seal is airtight. The oxygen source is connected, and the CPAP system begins delivering positive airway pressure.

### Step 2: Simultaneous ETCO2 Monitoring

As the CPAP system administers respiratory therapy, the integrated ETCO2 sensor begins measuring exhaled carbon dioxide levels. The sensor captures CO2 during exhalation and transmits the data to a connected monitoring device, providing real-time feedback on the patient's ventilatory status.

### Step 3: Data Analysis and Alert Mechanisms

The monitoring device analyzes the ETCO2 data, comparing the measured values to established clinical thresholds. If abnormal levels of ETCO2 are detected—indicative of respiratory failure, hypoventilation, or other critical conditions—the system alerts the healthcare provider. This allows for immediate intervention without interrupting CPAP therapy, thus optimizing patient outcomes.

### Step 4: Adjustment and Maintenance of CPAP Therapy

The mask is designed to be easily adjusted, allowing providers to fine-tune the fit and pressure settings based on the patient's clinical response. The system's architecture ensures that the ETCO2 sensors remain fully operational throughout, providing continuous monitoring even if the mask requires repositioning.

### Advantages and Improvements Over Existing Systems

Integrated Functionality: By combining CPAP therapy and ETCO2 monitoring in a single device, the system streamlines patient care, eliminating the need for multiple masks or separate oxygen sources.

Improved Efficiency: The system's simplified design reduces the number of steps required to administer care, decreasing setup time and allowing providers to focus on delivering treatment more effectively.

Enhanced Patient Comfort: The ergonomic design and reduced bulk of the mask contribute to a more comfortable patient experience, reducing anxiety and increasing tolerance for respiratory support.

Real-Time Monitoring: Continuous ETCO2 monitoring during CPAP therapy provides valuable insight into the patient's respiratory function, enabling faster clinical decision-making in critical situations.

Cost-Effectiveness: The integration of two systems into one device reduces the need for additional equipment purchases, saving healthcare providers and EMS agencies significant costs over time.

Simplified Training: The system's intuitive design makes it easier for healthcare providers to learn how to apply and use the mask effectively, reducing the need for extensive training.

### DETAILED DESCRIPTION OF FIGURES

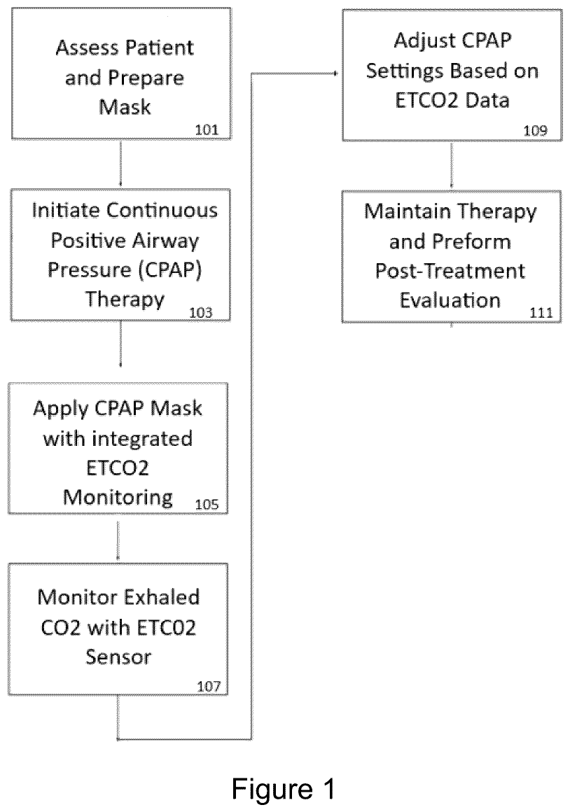

FIG. 1 provides a detailed sequence of steps involved in the administration of Continuous Positive Airway Pressure (CPAP) therapy using a specialized CPAP mask with integrated end-tidal carbon dioxide (ETCO2) monitoring. This figure outlines the essential procedures from patient assessment through to post-treatment evaluation, emphasizing the integration of ETCO2 monitoring to enhance therapeutic outcomes. Each step is sequentially organized to ensure clarity in the workflow:

FIG. 1.101: Assess Patient and Prepare Mask—In this initial step, the healthcare provider assesses the patient's respiratory status to determine the need for CPAP therapy. The CPAP mask with integrated ETCO2 monitoring is prepared for use by ensuring that all components, such as the oxygen source, sensor, and data transmission modules, are functional and properly connected.

FIG. 1.103: Initiate Continuous Positive Airway Pressure (CPAP) Therapy—The CPAP therapy is initiated by connecting the mask to a single oxygen source, which delivers continuous positive airway pressure. This maintains the patient's airway open and ensures proper ventilation. The mask's design ensures a minimal number of tubing sections to avoid disruption of the seal, enhancing the efficacy of the CPAP therapy.

FIG. 1.105: Apply CPAP Mask with Integrated ETCO2 Monitoring—Once the mask is prepared, it is applied to the patient's face, covering the nose and mouth to create a secure, airtight seal. The mask's adjustable straps are fastened to ensure comfort and stability, and to monitor the exhaled carbon dioxide without obstructing airflow.

FIG. 1.107: Monitor Exhaled CO2 with ETCO2 Sensor—As the CPAP therapy is underway, the integrated ETCO2 sensor continuously monitors the patient's exhaled carbon dioxide levels. The sensor provides real-time data on CO2 concentration during both the inspiratory and expiratory phases of the breathing cycle. This data is crucial for evaluating the patient's ventilatory status and detecting any abnormalities.

FIG. 1.109: Adjust CPAP Settings Based on ETCO2 Data—Based on the ETCO2 data analysis, healthcare providers can adjust the CPAP therapy settings, such as oxygen flow rate or pressure levels, to optimize patient ventilation. This step ensures that the therapy is tailored to the patient's specific respiratory needs, improving treatment outcomes.

FIG. 1.111: Maintain Therapy and Perform Post-Treatment Evaluation—The healthcare provider continues to monitor the patient's condition while maintaining the CPAP therapy. Following the treatment, a post-therapy evaluation is conducted to assess the effectiveness of the CPAP and ETCO2 monitoring. The mask is removed and disposed of as masks are only single use (with the exception of the defibrillation/cardiac monitor, which are cleaned and not single use).

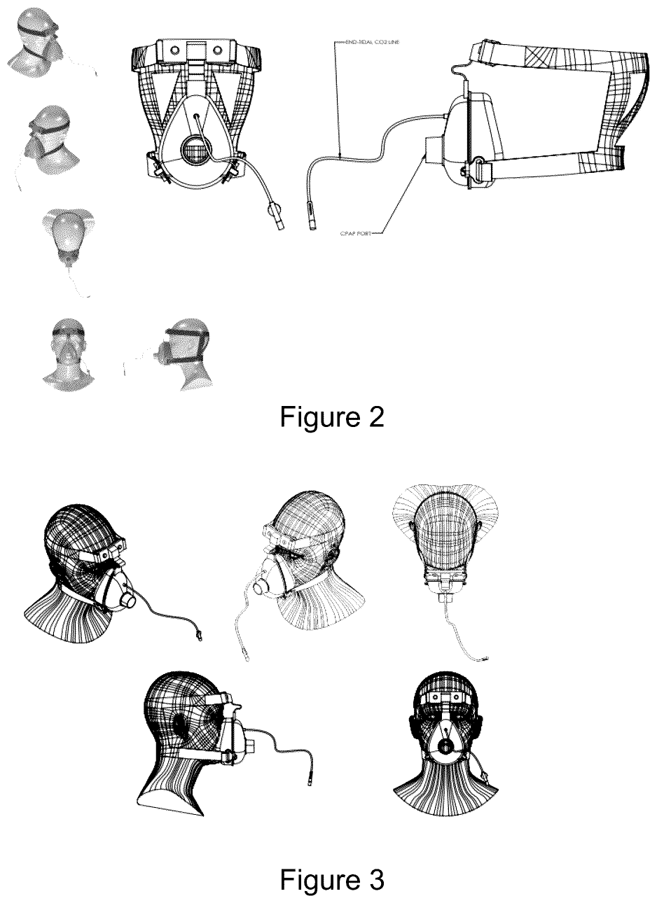

FIG. 2 illustrates the CPAP mask of the present invention. The tubing system has been optimized to minimize dead space and maximize the efficiency of airflow and gas exchange. By eliminating the extra tubing associated with standalone ETCO2 systems, the mask ensures a tight seal, which is crucial for maintaining continuous positive airway pressure. The tubing is constructed from medical-grade materials resistant to kinking, compression, and wear, ensuring uninterrupted oxygen delivery even in high-stress environments such as emergency transport.

FIG. 3 illustrates a user wearing the CPAP mask of the present invention. The design of the CPAP mask incorporates advanced ergonomic considerations to optimize patient comfort and provider usability. The facial attachment components are made from biocompatible, hypoallergenic materials to prevent irritation, even during prolonged use. The mask is contoured to create an airtight seal around the nose and mouth, eliminating leakage, which is critical for the efficacy of CPAP therapy. The attachment mechanism includes adjustable straps that are easy to secure yet maintain stability, ensuring proper alignment and consistent application of pressure without the need for frequent adjustments.

What is claimed is:

1. A continuous positive airway pressure (CPAP) mask with integrated End-Tidal Carbon Dioxide (ETCO2) monitoring, comprising:
a. A CPAP mask body designed to create an airtight seal around the nose and mouth of a patient, said mask being configured to deliver continuous positive airway pressure to maintain airway patency;
b. Wherein the mask is Configured to measure the concentration of exhaled carbon dioxide in real-time, without interfering with the CPAP function;
c. A data transmission module operatively connected to said ETCO2 sensor, configured to transmit ETCO2 data via wired connections to an external monitoring device for continuous respiratory monitoring;
d. A tubing system operatively connected to a single oxygen source, said system designed to minimize dead space and facilitate the delivery of oxygen while maintaining the integrity of the mask seal;
e. A user-adjustable fastening mechanism that secures the mask to the patient's face, ensuring a consistent application of pressure without requiring frequent readjustment.

2. The CPAP mask of claim 1, wherein said ETCO2 sensor is a capnography sensor configured to provide quantitative analysis of the patient's exhaled carbon dioxide levels.

3. The CPAP mask of claim 1, wherein said mask is constructed from hypoallergenic, biocompatible materials designed to prevent skin irritation during prolonged use.

4. The CPAP mask of claim 1, wherein said tubing system is designed to reduce airflow obstructions and maintain uninterrupted positive airway pressure by eliminating redundant or unnecessary tubing associated with traditional dual-mask systems.

5. A method for providing continuous positive airway pressure (CPAP) therapy while monitoring End-Tidal Carbon Dioxide (ETCO2) levels in real time, comprising the steps of:
a. Applying a CPAP mask with integrated ETCO2 monitoring to a patient in respiratory distress, said mask creating an airtight seal around the patient's nose and mouth
b. Delivering continuous positive airway pressure through the mask using a single oxygen source, ensuring that the patient's airway remains open during inhalation and exhalation;
c. Monitoring exhaled carbon dioxide levels through an integrated ETCO2 sensor wherein said sensor captures exhaled gas directly from the patient's airway;
d. Transmitting real-time ETCO2 data to a connected external monitoring device for continuous assessment of the patient's respiratory function; \
e. Adjusting CPAP settings based on ETCO2 data to optimize the patient's ventilation status and overall respiratory support.

6. The method of claim 5, further comprising the step of alerting healthcare providers if ETCO2 levels fall outside pre-determined clinical thresholds, enabling immediate intervention during CPAP therapy.

7. The method of claim 5, wherein said ETCO2 sensor is configured to continuously monitor both the inspiratory and expiratory phases of the patient's respiratory cycle.

8. The method of claim 5, wherein said CPAP mask reduces the number of oxygen sources and tubing connections required for respiratory support by integrating CPAP and ETCO2 monitoring into a single device.

9. A system for delivering continuous positive airway pressure (CPAP) and monitoring End-Tidal Carbon Dioxide (ETCO2) levels, comprising:
a. A CPAP mask configured to create an airtight seal over a patient's nose and mouth for administering respiratory support through continuous positive airway pressure;
b. An ETCO2 monitoring component integrated said CPAP mask, wherein said component is capable of detecting exhaled carbon dioxide levels in real-time during CPAP therapy;
c. A data processing and transmission unit configured to receive ETCO2 data from the monitoring component and transmit said data to an external monitoring system;
d. A single oxygen source operatively connected to said CPAP mask, wherein said oxygen source delivers pressurized oxygen through a simplified tubing system to ensure consistent airflow while minimizing the complexity of the system
e. A feedback mechanism operatively connected to the data processing unit, wherein said mechanism provides continuous feedback on the patient's ventilatory status, allowing for adjustments to be made to CPAP therapy as needed.

10. The system of claim 9, wherein the ETCO2 monitoring component uses capnography technology to provide quantitative CO2 measurements.

11. The system of claim 9, wherein the CPAP mask and ETCO2 components are designed for use in emergency medical settings, particularly in non-clinical environments, where rapid deployment and ease of application are critical.

12. The system of claim 9, further comprising user-adjustable fastening elements that allow medical providers to quickly secure the mask in place and ensure optimal comfort and fit for a wide range of patients.
